(19) United States
(12) Patent Application Publication
(10) Pub. No.: US 2017/0260254 A1
(43) Pub. Date: Sep. 14, 2017

(54) METHOD FOR PRODUCING VARIANTS HAVING AN FC WITH IMPROVED SIALYLATION

(71) Applicant: LABORATOIRE FRANCAIS DU FRACTIIONNEMENT ET DES BIOTECHNOLOGIES, Les Ulis (FR)

(72) Inventors: Celine MONNET, Lamebersart (FR); Alexandre FONTAYNE, La Madeleine (FR)

(21) Appl. No.: 15/500,105

(22) PCT Filed: Jul. 31, 2015
(86) PCT No.: PCT/FR2015/052123

§ 371 Date: Jan. 30, 2017

(30) Foreign Application Priority Data
Aug. 1, 2014 (FR) 14 57504

(51) Int. Cl. C07K 16/00; C07K 16/34; C07K 16/28

(52) U.S. Cl. CPC C07K 16/00; C07K 16/2887; C07K 16/34; C07K 2317/41; C07K 2317/52; C07K 2317/71; C07K 2317/94

(57) ABSTRACT

The present invention relates to a method for increasing the sialylation of an Fc fragment, including a mutation step of at least one amino acid selected from among the amino acids in positions 240 to 243, 258 to 267 and 290 to 305 of the Fc fragment, the numbering being that of the EU index or equivalent in Kabat. The present invention also relates to a method for producing a variant of a parent polypeptide including an Fc fragment, the variant having improved sialylation of the Fc fragment relatively to the parent polypeptide, which includes a mutation step of at least one amino acid selected from among the amino acids in positions 240 to 243, 258 to 267 and 290 to 305 of the Fc fragment, the numbering being that of the EU index or equivalent in Kabat.

The present invention relates to a method for increasing the sialylation of a fragment Fc, comprising a mutation step of at least one amino acid selected from the amino acids in positions 240 to 243, 258 to 267 and 290 to 305 of said fragment Fc, the numbering being that of the EU index or equivalent in Kabat.

Monoclonal antibodies are used today as therapeutic agents for treating a variety of pathologies, including cancers, auto-immune diseases, chronic inflammatory diseases, graft rejection, infectious diseases and cardio-vascular diseases. They therefore form a major therapeutic challenge. A number of them is already marketed and a still increasing proportion is being developed as clinical trials. However, there exists a significant need for optimizing the structural and functional properties of antibodies, in order to control the secondary effects thereof.

One of the critical questions in the use of monoclonal antibodies in therapy is their persistence in the blood flow. The clearance of the antibody directly affects the efficiency of the treatment, and therefore the frequency and the amount of the administration of the drug, which may cause undesirable effects in the patient.

Immunoglobulins of isotype G (IgG) forms the most frequent class of immunoglobulins in humans and also the most used in therapy. Different experiments of specific mutagenesis in the constant region (Fc) of mice IgGs have given the possibility of identifying certain critical amino acid residues involved, for some of them, in the interaction between the IgGs and the FcRn (Kim et al., 1994, Eur J Immunol.; 24:2429-34; Kim et al., 1994, Eur J Immunol.; 24: 542-8; Medesan et al., 1996, Eur J Immunol.; 26:2533-6; Medesan et al, 1997, J Immunol; 158:2211-7). Studies have more recently been conducted in humans (Shields et al., 2001, J. Biol. Chem.; 276: 6591-6604).

However, there exists always a need for finding antibodies, or antibody fragments, having an improved half-life, and having interesting biological properties.

The present invention provides means for obtaining a variant of a parent polypeptide comprising a fragment Fc with optimized sialylation. This optimized sialylation, i.e. improved, notably gives the variant an increased half-life, as well as optimized anti-inflammatory properties relatively to a parent polypeptide.

The term of [symbol] half-life [symbol] refers to a biological half-life of a polypeptide of interest in the blood stream of a given animal, and is represented by the time required for removing from the blood stream and/or from other tissues of the animal half of the amount present in the blood stream of the animal.

Indeed, surprisingly, the inventors discovered that a mutated fragment Fc on a specific position, close to the N-glycosylation site, has a strongly increased sialylation relatively to the non-mutated fragment Fc. This thus allows an increase in the properties of interest of the fragment Fc, and notably of its half-life. This may further allow an increase in its anti-inflammatory activity.

The object of the present invention is therefore a method for increasing sialylation of an Fc fragment, comprising a step for mutation of at least one amino acid selected from among the amino acids in positions 240 to 243, 258 to 267 and 290 to 305 of said Fc fragment, the numbering being that of the EU index or equivalent in Kabat.

Preferably, said method for increasing the sialylation of a Fc fragment comprises:
a mutation step of at least one amino acid selected from among the amino acids in positions 240 to 243, 258 to 267 and 290 to 305 of said Fc fragment, the numbering being that of the EU index or equivalent in Kabat, and then
a step for analyzing the sialylation of the obtained fragment Fc.

The object of the present invention is also a method for producing a variant of a parent polypeptide comprising an Fc fragment, said variant having improved sialylation of said Fc fragment relatively to the sialylation of the Fc fragment of the parent polypeptide, which comprises a mutation step of at least one amino acid selected from among the amino acids in positions 240 to 243, 258 to 267 and 290 to 305 of said Fc fragment, the numbering being that of the EU index or equivalent in Kabat.

Preferably, said method for producing a variant of a parent polypeptide comprising an Fc fragment comprises:
a mutation step of at least one amino acid selected from among the amino acids in positions 240 to 243, 258 to 267 and 290 to 305 of said Fc fragment, the numbering being that of the EU index or equivalent in Kabat, and then
a step for analyzing the sialylation of the obtained fragment Fc.

Preferably, said method for producing a variant of a parent polypeptide comprising an Fc fragment is such that the variant has at least one effector activity mediated by said fragment Fc reduced relatively to the effector activity of the parent polypeptide.

By [symbol] increase in the sialylation [symbol] or [symbol] improved sialylation [symbol] , is meant that the sialylation of the obtained protein is increased by at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, relatively to the sialylation of said Fc fragment before the mutation step of said fragment Fc of the parent polypeptide.

The sialylation of a protein is a well-known glycosylation mechanism (see notably Essentials of Glycobiology, 2^{nd} edition, Varki et al, 2009). It corresponds to an addition, through a covalent bond, of at least one sialic acid (i.e. N-acetylneuraminic acid and derivatives thereof, such as N-glycosylneuraminic acid, N-acetylglycoylneuraminic acid) in the glycosylated chain of the protein.

As used here, the terms of [symbol] protein [symbol] and [symbol] polypeptide [symbol] are used here interchangeably and refer to a sequence of at least two amino acids bound covalently, including proteins, polypeptides, oligopeptides and peptides.

The terms of [symbol] protein [symbol] and [symbol] polypeptide [symbol] notably include antibodies or immunoglobulins, notably entire, monoclonal, multi-specific, bi-specific, dual-specific, synthetic, chimeric, humanized, human, immunoglobulins, fusion proteins with immunoglobulins, conjugate antibodies and fragments thereof.

The terms of [symbol] protein [symbol] and [symbol] polypeptide [symbol] also include Fc polypeptides defined by a polypeptide comprising all or part of a region Fc, notably isolated Fc fragments, conjugate Fc, multimeric Fcs and fusion proteins with an Fc fragment.

By [symbol] Fc fragment [symbol] or [symbol] Fc region [symbol] , is meant the constant region of an immunoglobulin of a total length except for the first domain of constant immunoglobulin region (i.e. CH1-CL). Thus the fragment Fc refers to a homodimer, each monomer comprising the last two constant domains of IgAs, IgDs, IgGs (i.e. CH2 and CH3), or the three last constant domains of IgEs and IgMs (i.e. CH2, CH3 and CH4), and the N-terminal flexible hinge region of these domains. The fragment Fc then extends from IgA or IgM, may comprise the chain J. Preferably, an Fc fragment of a IgG1 is used in the present invention, which consists of the N-terminal flexible hinge and the domains CH2-CH3, i.e. the portion from the amino acid C226 as far as the C-terminal end, the numbering being indicated according to the EU index or equivalent in Kabat. Preferably, an Fc fragment of a human IgG1 is used (i.e. the amino acids 226 to 447 according to the EU index or equivalent in Kabat). In this case, the lower hinge refers to the positions 226 to 230, the domain CH2 refers to the positions 231 to 340 and the CH3 domain refers to the positions 341-447 according to the EU index or equivalent in Kabat. The fragment Fc used according to the invention may further comprise a portion of the upper hinge region, upstream from the position 226. In this case, preferably, a fragment Fc of a human IgG1 is used, comprising a portion of the region located between the positions 216 to 226 (according to the EU index). In this case, the fragment Fc of a human IgG1 refers to the portion from the amino acid 216, 217, 218, 219, 220, 221, 222, 223, 224 or 225 as far as the C-terminal end.

The definition of an [symbol] Fc fragment [symbol] includes an scFc fragment for [symbol] single chain Fc [symbol] . By [symbol] scFc fragment [symbol] , is meant a simple chain fragment Fc, obtained by genetic fusion of two monomers Fc connected through a polypeptide linker. The scFc is naturally folded-back into a functional dimeric Fc region. Preferably, the Fc fragment used within the scope of the invention is selected from among the Fc fragment of an IgG1 or IgG2. Still preferably, the fragment Fc used is the fragment Fc of an IgG1.

In the present application, the numbering of the residues of the Fc fragment is that of the EU index or equivalent in Kabat (Sequences of Proteins of Immunological Interest, 5th Ed. Public Health Service, National Institutes of Health, Bethesda, Md. (1991)).

In the context of the present invention, the parent polypeptide comprises an Fc fragment, called [symbol] parent Fc fragment [symbol] .

By [symbol] mutation of an amino acid [symbol] is meant a change in the sequence of amino acids of a polypeptide. A mutation is notably selected from among a substitution, an insertion and a deletion. By [symbol] substitution [symbol] , is meant the replacement of one or several amino acids, at a particular position in a sequence of a parent polypeptide, with the same number of other amino acids. Preferably, the substitution is point like, i.e. it only relates to a single amino acid. For example, the substitution N434S refers to a variant of a parent polypeptide, in which the asparagine in position 434 of the Fc fragment according to the EU index or equivalent in Kabat is replaced with serine. By [symbol] insertion [symbol] , is meant the addition of at least one amino acid at a particular position in a parent polypeptide sequence. For example, the insertion G>235-236 refers to an insertion of glycine between the positions 235 and 236. By [symbol] deletion [symbol] , is meant the removal of at least one amino acid at a particular position in a parent polypeptide sequence. For example, E294del refers to the suppression of glutamic acid in the position 294; such a deletion is called De1294.

By [symbol] parent polypeptide [symbol] , is meant a non-modified polypeptide which is then modified for generating a variant. Said parent polypeptide may be a polypeptide of natural origin, a variant of a polypeptide of natural origin, a modified version of a natural polypeptide or a synthetic polypeptide. Preferably, the parent polypeptide comprises a fragment Fc selected from among the Fc fragments of the wild type, their fragments and their mutants. Therefore, the parent polypeptide may optionally comprise pre-existing modifications of amino acids in the fragment Fc relatively to the fragments Fc of the wild type. Thus preferably, the fragment Fc of the parent polypeptide already comprises at least one additional mutation (i.e. a pre-existing modification), preferably selected from among P230S, T256N, V2591, N315D, A330V, N361D, A378V, 5383N, M428L, N434Y. Preferably, the Fc fragment of the parent polypeptide comprises at least one combination of additional mutations selected from among P230S/N315D/M428L/N434Y, T256N/A378V/S383N/N434Y, V2591/N315D/N434Y and N315D/A330V/N 361 D/A378V/N434Y.

Preferably, according to a first alternative, the parent polypeptide consists in a fragment Fc, and preferably an entire Fc fragment.

Preferably, according to a second alternative, the parent polypeptide consists in a sequence of amino acids fused at an N- or C-terminal to a fragment Fc. In this case, advantageously, the parent polypeptide is an antibody, a fusion Fc or a conjugate Fc polypeptide.

Preferably, the fragment Fc of the parent polypeptide is selected from among the sequences SEQ ID NO: 1, 2, 3, 4 and 5. Preferably, the fragment Fc of the parent polypeptide has the sequence SEQ ID NO: 1.

The sequences represented in SEQ ID NO: 1, 2, 3, 4 and 5 are without any hinge region at an N-terminal.

The sequences represented in SEQ ID NO: 6, 7, 8, 9 and 10 respectively correspond to the sequences represented in SEQ ID NO: 1, 2, 3, 4 and 5 with their hinge regions in an N-terminal position. Also, in a particular embodiment, the fragment Fc of the parent polypeptide is selected from among the sequences SEQ ID NO: 6, 7, 8, 9 and 10. Preferably, the fragment Fc of the parent polypeptide has a sequence corresponding to the positions 1-232, 2-232, 3-232, 4-232, 5-232, 6-232, 7-232, 8-232, 9-232, 10-232 or 11-232 of the sequence SEQ ID NO: 6.

Alternatively, the parent polypeptide consists in an immunoglobulin, an antibody or further in a sequence of amino acids fused at the N- or C-terminal to an antibody or an immunoglobulin.

By [symbol] variant [symbol] , is meant a polypeptide sequence which is different from the sequence of the parent polypeptide by at least one modification of an amino acid. Preferably, the sequence of the variant has at least 80% identity with the sequence of the parent polypeptide, and more preferentially at least 90%, 91%, 92%, 93%, 94%, 95%, 96%, 97%, 98%, 99% or 99.5% of identity. By [symbol] percentage of identity [symbol] between two sequences of amino acids in the sense of the present invention, is meant to refer to a percentage of amino acid residues which are identical between both sequences to be compared, obtained after the best alignment, this percentage being purely statistical and the differences between both sequences being randomly distributed and over the whole of their length. By [symbol] best alignment [symbol] or [symbol] optimum alignment [symbol] , is meant the alignment for which the identity percentage determined as hereafter is higher. The comparisons of sequences between two sequences of amino acids are traditionally made by comparing these sequences after having aligned them in an optimum way, said comparison being made per segment or per [symbol] comparison window [symbol] for identifying and comparing the local sequence similarity regions. The optimum alignment of the sequences for the comparison may be made further manually, by means of the local homology algorithm of Smith and Waterman (1981, J. Mol Evol., 18:38-46), by means of the local homology algorithm of Neddleman and Wunsch (1970), by means of the similarity search method of Pearson and Lipman (1988, PNAS, 85: 2444-2448), by means of computer software packages using these algorithms (GAP, BESTFIT, BLAST P, BLAST N, FASTA and TFASTA in the Wisconsin Genetics Software Package, Genetics Computer Group, 575 Science Dr., Madison, Wisc.).

In preferred embodiments, the parent polypeptide is an immunoglobulin or an antibody, preferably a IgG, and the variant according to the invention is then selected from among the variants of IgG. More preferentially, the variant according to the invention is selected from among the variants of human IgG1, IgG2, IgG3 and IgG4.

Preferably, the method for producing a variant according to the invention or the method for increasing the sialylation according to the invention comprises a mutation performed on at least one amino acid of the Fc fragment located in positions 240, 241, 242, 243, 258, 259, 260, 261, 262, 263, 264, 265, 266, 267, 290, 291, 292, 293, 294, 295, 296, 297, 298, 299, 300, 301, 302, 303, 304 or 305, the numbering being that of the EU index or equivalent in Kabat. Preferably, the method for producing the variant or the method for increasing the sialylation according to the invention comprises at least one mutation on the Fc fragment selected from among V262del, V263F, V263K, V263W, V264K, V264P, D265A, D265E, D265G, D265L, D265S, D265V, V266A, V266P, V266S, V266T, S267N, S267P, S267R, S267W, P291C, P291V, P291Y, P291W,

R292A, R292del, R292T, R292V, R292Y, E293F, E293P, E293W, E293Y, E294del, E294D, E294N, E294W, E294F, Q295D, Q295del, Q295F, Q295G, Q295K, Q295N, Q295R, Q295W, Y296A, Y296C, Y296del, Y296E, Y296G, Y296Q, Y296R, Y296V, S298del, S298E, S298F, S298G, S298L, S298M, S298N, S298P, S298R, S298T, S298W, S298Y, Y300D, Y300del, Y300G, Y300N, Y300P, Y300R, Y300S, R301A,

R301F, R301G, R301H, R3011, R301K, R301Q, R301V, R301W, R301Y, V302del, V302A, V302F, V302G, V302P, V303A, V303C, V303P, V303L, V303S, V303Y, S304C, S304M, S304Q, S304T, V305F and V305L, the numbering being that of the EU index or equivalent in Kabat.

Preferably, the invention aims at a method for increasing the sialylation of an Fc fragment, comprising a mutation step of at least one amino acid selected from among the amino acids in positions 240 to 243, 258 to 267 and 290 to 305 of said fragment Fc, the numbering being that of the EU index or equivalent in Kabat, except for the amino acids in positions 262 and 264.

The invention also preferably aims at a method for producing a variant of a parent polypeptide comprising an Fc fragment, said variant having improved sialylation of said Fc fragment relatively to the parent polypeptide, which comprises a mutation step of at least one amino acid selected from among the amino acids in positions 240 to 243, 258 to 267 and 290 to 305 of said Fc fragment, the numbering being that of the EU index or equivalent in Kabat, and wherein said mutation step does not concern any of the amino acids in positions 262 or 264.

Thus, according to these preferential embodiments, the mutation is carried out on the amino acid of the Fc fragment located in position 240, 241, 242, 243, 258, 259, 260, 261, 263, 265, 266, 267, 290, 291, 292, 293, 294, 295, 296, 298, 299, 300, 301, 302, 303, 304 or 305. More preferentially, the mutation is carried out on the amino acid of the Fc fragment located in positions 293 or 294, the numbering being that of the EU index or equivalent in Kabat. According to a particular embodiment, the mutation is carried out on the two amino acids of the Fc fragment located in position 293 and in position 294, the numbering being that of the EU index or equivalent in Kabat.

The object of the present invention is also a method for producing a variant of a parent polypeptide comprising an Fc fragment, said variant having at least one effector activity mediated by said Fc fragment, reduced relatively to the effector activity of the parent polypeptide, said method comprising a mutation step of at least one amino acid selected from among the amino acids in positions 240 to 243, 258 to 267 and 290 to 305 of said Fc fragment, the numbering being that of the EU index or equivalent in Kabat.

By [symbol] effector activity mediated by the Fc fragment [symbol] , is notably meant the cell cytotoxicity depending on antibodies (ADCC or Antibody-Dependent Cell-mediated Cytotoxicity), the cytotoxicity dependent on the complement (CDC or Complement Dependent Cytotoxicity), cell phagocytosis depending on antibodies (ADCP), endocytosis activity or further the secretion of cytokines. Preferably, the effector activity mediated by the relevant Fc fragment in the invention is selected from cell cytotoxicity dependent on antibodies (ADCC), cytotoxicity depending on the complement (CDC) and cell phagocytosis dependent on antibodies (ADCP).

By [symbol] reduced [symbol] effector activity is meant a reduced or abolished effector activity. Thus, a variant of a parent polypeptide produced by a method according to the invention may have at least one of abolished effector activity mediated by the Fc fragment. Preferably, a variant of a parent polypeptide produced by a method according to the invention has a reduced effector activity mediated by the Fc region, relatively to that of the parent polypeptide, by at least 10%, preferably of at least 20%, 30%, 40%, 50%, 60%, 70%, 80%, or 90%.

In a particular embodiment, the invention provides a method for producing a variant of a parent polypeptide comprising an Fc fragment, said variant being without any effector activity mediated by said Fc fragment, comprising a mutation step of at least one amino acid selected from among the amino acids in positions 240 to 243, 258 to 267 and 290 to 305 of said fragment Fc, the numbering being that of the EU index or equivalent in Kabat.

Preferably according to this aspect, the invention provides a method for producing a variant of a parent polypeptide comprising an Fc fragment, said variant having at least one effector activity mediated by said Fc fragment, reduced relatively to the effector activity of the parent polypeptide, comprising a mutation step of at least one amino acid selected from among the amino acids in positions 240 to 243, 258 to 267 and 290 to 305 of said Fc fragment, the numbering being that of the EU index or equivalent in Kabat, and wherein the mutation step does not affect the amino acids in positions 262, 264, 293 or 294.

According to another aspect, the object of the present invention is a method for producing a variant of a parent polypeptide comprising an Fc fragment, said variant having an affinity mediated by said Fc fragment, reduced relatively to the affinity of the parent polypeptide, for at least one of the receptors of the Fc region (FcR), comprising a mutation step of at least one amino acid selected from among the amino acids in positions 240 to 243, 258 to 267 and 290 to 305 of said Fc fragment, the numbering being that of the EU index or equivalent in Kabat.

By [symbol] receptor of the Fc region [symbol] or [symbol] FcR [symbol] is notably meant C1q and the Fcy Receptors (FcyR). The [symbol] Fcy Receptors [symbol] or [symbol] FcyR [symbol] refer to the receptors of Immunoglobulins of the IgG type, called CD64 (FcyRI), CD32 (FcyRII), and CD16 (FcyRIII), in particular to the five expressed receptors FcyRIa, FcyRIIa, FcyRIIb, FcyRIIIa and FcyRIIIb. All are receptors which are activators of effector cells, except for the human FcyRIIb which is a receiver which is an inhibitor of the activation of immune cells (Muta T et al., Nature, 1994, 368:70-73).

The complement C1q is involved in the CDC activity.

The receptor FcgRIIIa (CD16a) is, as for it, involved in ADCC; it has a polymorphism V/F in position 158.

The receptor FcgRIIa (CD32a) is, as for it involved in the platelet activation and phagocytosis; it has a polymorphism H/R in position 131.

Finally, the receptor FcgRIIb (CD32b) is involved in the inhibition of the cell activity.

By [symbol] reduced [symbol] affinity, is meant a reduced or abolished affinity. Preferentially, the affinity is reduced, relatively to that of the parent polypeptide comprising the Fc fragment, by at least 10%, preferably by at least 20%, 30%, 40%, 50%, 60%, 70%, 80%, or 90%.

Preferably, the invention provides a method for producing a variant of a parent polypeptide comprising an Fc fragment, said variant having an affinity mediated by said Fc fragment, reduced relatively to the affinity of the parent polypeptide, for at least one of the receptors of the Fc region (FcR) selected from among the complement C1q and the receptors FcgRIIIa (CD16a), FcgRIIa (CD32a) and FcgRIIb (CD32b), comprising a mutation step of at least one amino acid selected from among the amino acids in positions 240 to 243, 258 to 267 and 290 to 305 of said Fc fragment, the numbering being that of the EU index or equivalent in Kabat.

Preferentially, the variant produced according to the invention has an affinity mediated by the Fc fragment, reduced relatively to that of the parent polypeptide, by at least 10%, preferably by at least 20%, 30%, 40%, 50%, 60%, 70%, 80%, or 90%. In other words, the affinity of the mutated Fc fragment for an FcR is less than that of the parent polypeptide. Preferably, the ratio of the affinity mediated by the Fc fragment of a variant according to the invention relatively to that of the parent polypeptide is less than 0.9, preferably less than 0.8, preferably less than 0.7, preferably less than 0.6, preferably less than 0.5, preferably less than 0.4, preferably less than 0.3, preferably less than 0.2, preferably less than 0.1. For example, the ratio of the affinity mediated by the Fc fragment of a variant according to the invention relatively to that of the parent polypeptide is less than 0.7. Still preferably, the ratio of the affinity mediated by the Fc fragment of a variant according to the invention relatively to that of the parent polypeptide is comprised between 0.9 and 0.1, preferably between 0.8 and 0.2, between 0.7 and 0.3, or between 0.6 and 0.4.

The affinity of a polypeptide comprising an Fc fragment for an FcR may be evaluated by methods well known to the prior art. For example, one skilled in the art may determine the affinity (Kd) by using surface plasmon resonance (SPR). Alternatively, one skilled in the art may carry out a suitable ELISA test. A suitable ELISA assay gives the possibility of comparing the binding forces of the parent Fc and of the mutated Fc. The specific signals detected from the mutated Fc and from the parent Fc are compared. The binding affinity may be equally determined by evaluating the entire polypeptides or by evaluating the regions Fc isolated from the latter.

According to a particular embodiment, a variant produced according to a method subject of the invention has an affinity mediated by said Fc fragment, reduced relatively to the affinity of the parent polypeptide, for the receiver FcgRIIIa (CD16a), and the receptor FcgRIIa (CD32a).

Preferably according to this other aspect, the invention provides a method for producing a variant of a parent polypeptide comprising an Fc fragment, said variant having an affinity mediated by said Fc fragment, reduced relatively to the affinity of the parent polypeptide, for at least one of the receptors of the Fc region (FcR), preferably selected from among the complement C1q and the receptors FcgRIIIa (CD16a), FcgRIIa (CD32a), and FcgRIIb (CD32b), and comprising a mutation step of at least one amino acid selected from among the amino acids in positions 240 to 243, 258 to 267 and 290 to 305 of said Fc fragment, the numbering being that of the EU index or equivalent in Kabat and in which the mutation step does not apply on any of the amino acids in positions 262, 264, 293 or 294.

According to a particular embodiment, the mutation is selected from among an insertion, a substitution, preferably point like, and a deletion, and is carried out on at least one amino acid located in position 240, 241, 242, 243, 258, 259, 260, 261, 263, 265, 266, 267, 290, 291, 292, 295, 296, 298, 299, 300, 301, 302, 303, 304 or 305, the numbering being that of the EU index or equivalent in Kabat.

Advantageously, a variant of a parent polypeptide comprising an Fc fragment produced according to the invention may have affinity for the receptor FcRn mediated by the Fc fragment, retained or increased, relatively to said affinity of the parent polypeptide. Preferably, the mutation(s) comprised by the variant according to the invention do(es) affect the affinity for the FcRn receptor mediated by the Fc fragment. In other words, preferably, the variant of a parent polypeptide comprising an Fc fragment produced according to the invention comprises one or several mutations which do not affect the affinity for the receptor FcRn mediated by the Fc fragment relatively to the affinity of the parent polypeptide.

By “FcRn” or “newborn receptor Fc” as used here, is meant a protein which is bound to the Fc region of the IgGs and is coded at least partly by a FcRn gene. The FcRn may be of any organism, including, but not being limited thereto, humans, mice, rats, rabbits and monkeys. As this is known in the technique, the functional FcRn protein comprises two polypeptides, often designated as a heavy chain and a lightweight chain protein. The lightweight chain is beta-2-microglobulin and the heavy chain is coded by the FcRn gene. Unless indicated otherwise here, FcRn or the FcRn protein refers to the complex of the chain a with beta-2-microglobulin. In humans, the gene coding for FcRn is called FCGRT.

The sequences described in the present application may be summarized as follows:

| SEQ ID NO: | Protein |
|---|---|
| 1 | Fc fragments of human IgG1 G1m1,17 (residues 226-447 |
| | according to the EU index or equivalent in Kabat) without |
| | any N-terminal hinge region. |
| 2 | Fc fragment of human IgG2 without any N-terminal hinge |
| | region. |
| 3 | Fc fragment of human IgG3 without any N-terminal hinge |
| | region. |
| 4 | Fc fragment of human IgG4 without any N-terminal hinge |
| | region. |
| 5 | Fc fragment of human IgG1 G1m3 without any N-terminal |
| | hinge region. |
| 6 | Fc fragment of human IgG1 G1m1,17 (residues 226-447 |
| | according to the EU index or equivalent in Kabat) with N- |
| | terminal hinge region. |
| 7 | Fc fragment of human IgG2 with N-terminal hinge region. |
| 8 | Fc fragment of human IgG3 with N-terminal hinge region. |
| 9 | Fc fragment of human IgG4 with N-terminal hinge region. |
| 10 | Fc fragment of human IgG1 G1m3 with N-terminal hinge |
| | region. |

More preferentially, the mutation step of the method for preparing the variant according to the invention is obtained as follows:
i) a nucleic sequence is provided, coding for the parent polypeptide comprising the Fc fragment;
ii) the nucleic sequence provided in i) is modified in order to obtain a nucleic sequence coding for the variant; and
iii) the nucleic sequence obtained in ii) is expressed in a host cell and the variant is recovered.

Such a mutation step is therefore carried out by using a nucleic sequence (polynucleotide or nucleotide sequence) coding for said parent polypeptide (step i)). The nucleic sequence coding for the parent polypeptide may be synthesized via a chemical route (Young L and Dong Q., 2004,-Nucleic Acids Res., Apr 1 5;32(7), Hoover, D. M. and Lubkowski, J. 2002, Nucleic Acids Res., 30, Villalobos A, et al., 2006. BMC Bioinformatics, Jun 6;7:285). The nucleotide sequence coding for the parent polypeptide may also be amplified by PCR by using suitable primers. The nucleotide sequence coding for the parent polypeptide may also be cloned in an expression vector. The DNA coding for such a parent polypeptide is inserted into an expression plasmid and inserted into an ad hoc cell line for its production (for example the line HEK-293 FreeStyle, the line YB2/O, or the line CHO), the thereby produced protein then being purified by chromatography.

These techniques are described in details in the reference manuals: Molecular cloning: a laboratory manual, 3^{rd} edition-Sambrook and Russel eds. (2001) and Current Protocols in Molecular Biology—Ausubel et al. eds (2007).

The nucleic sequence provided in i) (polynucleotide), which codes for the parent polypeptide is then modified in order to obtain a nucleic sequence coding for the variant. This is step ii).

This step is the mutation step strictly speaking. It may be carried out by any method known to the prior art, notably by directed mutagenesis or by random mutagenesis. Preferably, random mutagenesis as described in application WO02/038756 is used: this is the Mutagen technique. This technique uses a human mutase DNA, notably selected from among DNA polymerases β, η and τ. A step for selecting the mutants having retained the bond to the FcRn is required for retaining the mutants of interest.

Alternatively, the substitutions of amino acids are preferably carried out by directed mutagenesis, by the assembling PCR technique using degenerated oligonucleotides (see for example, Zoller and Smith, 1982, Nucl. Acids Res. 10:6487-6500; Kunkel, 1985, Proc. Nati. Acad. Sci USA 82;488).

Finally, in step iii), the nucleic sequence obtained in ii) is expressed in a host cell, and the thereby obtained variant is recovered.

The host cell may be selected from among prokaryotic or eukaryotic systems, for example bacterial cells but also yeast cells or animal cells, in particular mammal cells.

It is also possible to use insect cells or plant cells.

The preferred host cells are the rat line YB2/0, the hamster line CHO, in particular the CHO dhfr- and CHO Lec13, PER.C6™ (Crucell), HEK293, T1080, EB66, K562, NSO, SP2/0, BHK or COS lines. Still preferably, the rat line YB2/0 is used.

Alternatively, the host cells may be modified transgenic animal cells for producing the polypeptide in milk. In this case, the expression of a DNA sequence coding for the polypeptide according to the invention is controlled by a mammal casein promoter or a mammal lactoserum promoter, said promoter not naturally controlling the transcription of said gene, and the DNA sequence further containing a protein secretion sequence. The secretion sequence comprises a secretion signal interposed between the coding sequence and the promoter. The animal may thus be selected from sheep, goats, rabbits, ewes or cows.

The polynucleotide coding for the variant obtained in step ii) may also comprise optimized codons, notably for its expression in certain cells (step iii)). For example, said cells comprise the COS cells, the CHO cells, the HEK cells, the BHK cells, the PER.C6 cells, the HeLa cells, the NIH/3T3, 293 cells (ATCC # CRL1573), T2 cells, dendritic cells or monocytes. The codon optimization has the purpose of replacing the natural codons with codons for which the transfer RNAs (RNAt) bearing the amino acids are the most frequent in the relevant cell type. The fact of mobilizing the RNAt frequently encountered has the major advantage of increasing the translation speed of the messenger RNAs (RNAm) and therefore of increasing the final titre (Carton J M et al, Protein Expr Purif, 2007). The optimization of codons also acts on the prediction of the secondary structures of RNAm which may slow down the reading by the ribosomal complex. The optimization of codons also has an impact on the percentage of G/C which is directly related to the half-life of the ARNAms and therefore to their translation potential (Chechetkin, J. of Theoretical Biology 242, 2006 922-934).

The optimization of codons may be accomplished by substituting natural codons by using frequency tables of codons (codon Usage Table) for mammals and more particularly for Homo sapiens. There exist algorithms present on the internet and made available by providers of synthetic genes (DNA2.0, GeneArt, MWG, Genscript) which give the possibility of accomplishing this sequence optimization.

Preferably, the polynucleotide comprises optimized codons for its expression in HEK cells, such as HEK293 cells, CHO cells, or YB2/0 cells. More preferentially, the polynucleotide comprises optimized codons for its expression in YB2/0 cells. Alternatively, preferably, the polynucleotide comprises optimized codons for its expression in the cells of transgenic animals, preferably goats, rabbits, ewes or cows.

The variant obtained according to the invention may be combined with pharmaceutically acceptable excipients, and optionally matrices with prolonged release, like biodegradable polymers, in order to form a therapeutic composition. The pharmaceutical composition may be administered via an oral, sublinguale, sub-cutaneous, intramuscular, intravenous, intra-arterial, intrathecal, intra-ocular, intra-cerebral, transdermal, pulmonary, local or rectal route. The active ingredients either alone or associated with another active ingredient, may then be administered as a unit dosage form, mixed with conventional pharmaceutical carriers. Dosage unit forms comprise orally administered forms such as tablets, gelatin capsules, powders, granules and oral solutions or suspensions, sublingual and buccal administration forms, aerosols, sub-cutaneous, transdermal, topical, intraperitoneal, intramuscular, intravenous, sub-cutaneous, intrathecal implants, administration forms via an intranasal route and rectal administration forms.

Preferably, the pharmaceutical composition contains a pharmaceutically acceptable carrier acceptable for a formulation which may be injected. This may in particular be isotonic, sterile formulations, saline solutions (with monosodium or disodium phosphate, sodium, potassium, calcium or magnesium chloride and the like, or mixtures of such salts), or freeze-dried compositions, which, upon adding sterilized water or physiological saline depending on the cases, allows the formation of injectable solutes.

The suitable pharmaceutical forms for an injectable use comprise sterile aqueous solutions or dispersions, oily formulations, including sesame oil, groundnut oil, and sterile powders for extemporaneous preparation of sterile injectable solutions or dispersions. In every case, the form has to be sterile and has to be fluid in so far that it has to be injected through a syringe. It has to be stable under the manufacturing and storage conditions and has to be preserved against the contaminating action of micro-organisms, such as bacteria and fungi.

The dispersions according to the invention may be prepared in glycerol, liquid polyethyleneglycols or mixtures thereof, or in oils. Under the normal storage and use conditions, these preparations contain a preservative for preventing the growth of micro-organisms.

The pharmaceutically acceptable carrier may be a solvent or a dispersion medium for example containing water, ethanol, a polyol (for example glycerol, propylene glycol, polyethylene glycol and the like), suitable mixtures of the latter and/or vegetable oils. The suitable fluidity may be maintained, for example by using a surfactant such as lecithin. Preventing the action of micro-organisms may be caused by diverse antibacterial and antifungal agents, for example parabens, chlorobutanol, phenol, sorbic acid or further thimerosal. In many cases, it will be preferable to include isotonic agents, for example, sugars or sodium chloride. The extended absorption of injectable compositions may be caused by the use in the compositions of agents delaying absorption, for example aluminium monostearate or gelatin.

The sterile injectable solutions are prepared by incorporating active substances in a required amount in the suitable solvent with several of the other ingredients listed above, if necessary followed by sterilization with filtration. As a rule, the dispersions are prepared by incorporating the diverse sterilized active ingredients in a sterile carrier which contains the basic dispersion medium and the other ingredients required from among those listed above. In the case of sterile powders for the preparation of sterile injectable solutions, the preferred preparation methods are the drying in vacuo and freeze-drying. During the formulation, the solutions will be administered in a compatible way with the dosage formulation and in a therapeutically effective amount. The formulations are easily administered in a variety of galenic forms, such as the injectable solutions described above, but drug releasing capsules and similar capsules may also be used. For parenteral administration in an aqueous solution for example, the solution has to be suitably buffered and the liquid diluent made isotonic with sufficient amount of saline or glucose solution. These particular aqueous solutions are particularly suitable for intravenous, intramuscular, sub-cutaneous and intraperitoneal administration. In this respect, sterile aqueous media which may be used are known to one skilled in the art. For example, a dose may be dissolved in 1 ml of isotonic NaCl solution and then added to 1000 ml of a suitable liquid, or injected on the proposed site of the perfusion. Certain dosage variations will necessarily have to occur depending on the condition of the treated subject.

The pharmaceutical composition of the invention may be formulated in a therapeutic mixture comprising about 0.0001 to 1.0 milligrams, i.e. about 0.001 to 0.1 milligrams, i.e. about from 0.1 to 1.0 milligrams, or even about 10 milligrams per dose or more.

Multiple doses may also be administered. The specific therapeutically effective dose level for a particular patient will depend on a variety of factors, including the disorder which is treated and the seriousness of the disease, the activity of the specific compound used, the specific composition used, the age, the body weight, the general health, the gender and the food diet of the patient, the moment of the administration, the administration route, the excretion level of the specific compound used, the duration of the treatment, or further the drugs used in parallel.

### FIGURES

FIG. 1: alignments of native human IgG1 sequences referring to the positions 216 to 447 according to the EU index:

FIG. 1 shows alignments of native human IgG1 sequences referring to the positions 216 to 447 (according to the EU index) with the corresponding sequences of human IgG2 (SEQ ID NO: 2 and 7), human IgG3 (SEQ ID NO: 3 and 8) and human IgG4 (SEQ ID NO: 4 and 9). The IgG1 sequences refer to the allotype G1m1,17 (SEQ ID NO: 1 and 6) and to the allotype G1m3 (SEQ ID NO: 5 and 10). The [symbol] lower hinge CH2-CH3 [symbol] IgG1 domain begins at position 226 (see arrow). The CH2 domain is highlighted in grey and the CH3 domain is in italics.

FIG. 2: Half-life of anti-CD20 and anti-Rhesus D antibodies produced in YB2/0: The persistence of immunoglobulins in the serum of transgenic mice for human FcRn was evaluated; Two antigenic specificities were tested; the anti-CD20 IgGs and the deleted anti-RhD IgGs in position 294 were tested as a comparison with the corresponding IgG WT.
A) Illustrates the time-dependent change in the concentration of plasma IgGs;
B) Ilustrates the half-life observed for both IgGs deleted in position 294 and of the IgG WTs.

### EXAMPLES

The following examples are given with view for illustrating diverse embodiments of the invention.

### Example 1

### Production of Variants Deleted in Position 294

The inventors analyzed the sialylation of several variants according to the invention, notably deleted in position 294 (EU index or equivalent in Kabat). From among the analyzed Del294 variants, several variants comprise a combination of additional mutations from among the combinations described for providing an optimized bond to the FcRn in patent application EP 0 233 500.

The identification and the obtaining of such [symbol] FcRn optimized [symbol] variants, may be accomplished according to the methods described in the prior art, in particular in the

European patent application EP 0 233 500, which describes the obtaining of such mutants according to the so-called MutaGen™ Technique.

Typically, this method comprises the following steps:

### A/Building an Fc Bank

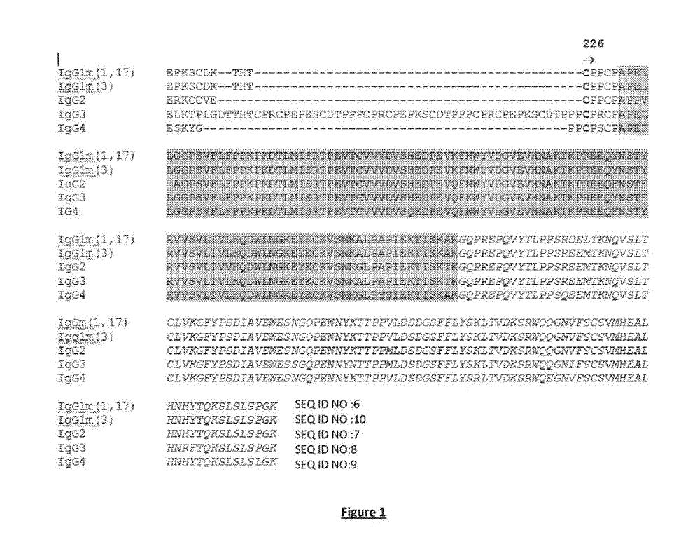

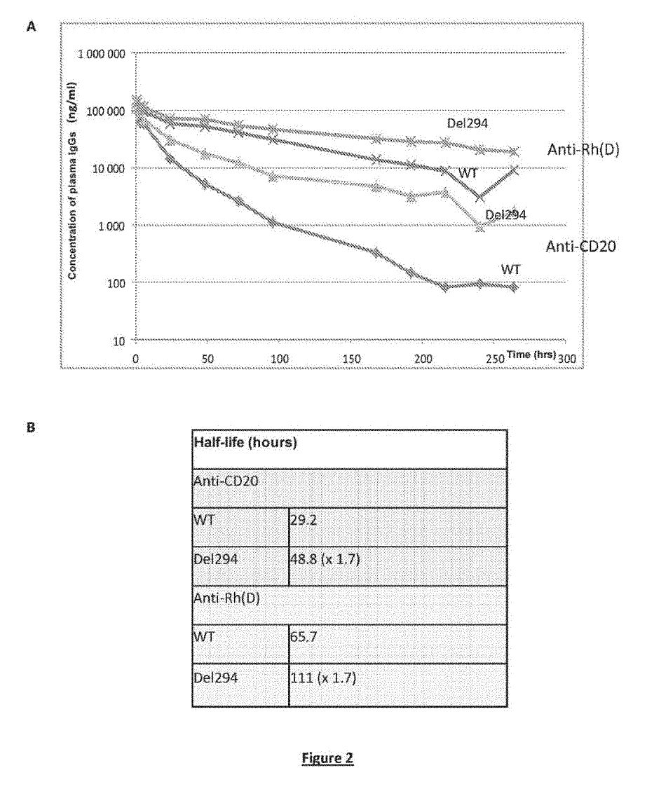

The human gene Fc coding for the residues 226 to 447 (according to the EU index of Kabat and illustrated in FIG. 1) derived from the heavy chain of a human IgG1 is cloned in a suitable vector, such as the phagemid vector pMG58 according to standard procedures well known to one skilled in the art.

### B/Mutagenesis

Several banks are then generated, according to the procedure described in WO 02/038756, which uses human polymerase DNAs of low reliability with the purpose of introducing random mutations homogenously on the entire target sequence. More specifically, three distinct mutases (pol β, η and τ) were used under different conditions for generating profiles of complementary mutations.

### C/Expression of the Fc Banks by Phage-Display and Selection of the Variants Having an Improved Bond to the Neonatal Receptor FcRn

The Fc banks are expressed by using the Phage-display technique according to standard procedures, for being used for selecting Fc fragments. The selection may be accomplished according to the detailed procedure in European patent application EP 2 233 500, notably by selection on FcRn in a solid or liquid phase, and then determination of the binding characteristics of the fragments to FcRn with ELISA.

### D/Production of Variants as an Entire Ig and Deletion in Position 294

Several combinations of [symbol] FcRn optimized [symbol] mutations were selected for being used as a base for the production of mutants deleted in position 294. The following combinations were selected:
N315D/A330V/N361D/A378V/N434Y (T5A-74)
T256N/A378V/S383N/N434Y (C6A-78)
V2591/N315D/N434Y (C6A-74)

### 1—Production of IgG Variants in HEK Cells

The Fc fragment sequence SEQ ID NO: 1 was cloned in a generic eukaryotic expression vector derived from pCEP4 (Invitrogen) and containing the heavy chain of an anti-CD20 chimeric antibody according to standard PCR procedures. The lightweight chain of this antibody was inserted into a similar pCEP4-derived vector. All the mutations of interest in the Fc fragment were inserted into the expression vector containing the anti-CD20 heavy chain by overlap PCR. For example, the variant 294Del was obtained by using two sets of primers adapted for integrating the deletion in position 294 on the heavy chain contained in the expression vector.

The thereby obtained fragments by PCR were associated and the resulting fragment was amplified by PCR by using standard procedures. The PCR product was purified on 1% agarose gels (w/v), digested with the suitable restriction enzymes and cloned in the expression vector of the anti-CD20 heavy chain.

The HEK 293 cells were co-transfected with the expression vectors of the lightweight chain and of the heavy chain of the anti-CD20 IgG in equimolar amounts according to standard procedures (Invitrogen). The cells were cultivated so as to produce antibodies in a transient way. The produced antibodies were able to be isolated and purified according to current techniques of the art, with view to their characterization.

### 2—Production of IgG Variants in YB2/0 Cells

The Fc variants were prepared in an entire IgG format in the cell line YB2/0 (ATCC, CRL-1662) with the anti-CD20 and anti-RhD specificity. For this, the heavy and lightweight chain of the IgGs were cloned in a bicistronic HKCD20 vector optimized for production in YB2/0. The production was made in stable pools of YB2/0 cells. The production steps by cell cultivation and cultivation for purifying the antibodies were carried out according to current techniques of the art, with view to their characterization.

The inventors verified that the deletion in position 294 did not have any significant impact on the binding to FcRn. The variants deleted in position 294 preserve their binding to FcRn relatively to the parent IgG (IgG WT or IgG comprising [symbol] FcRn optimized [symbol] mutations).

### Example 2

### Analysis of the Sialylation of Different Proteins

### Operating Method: Preparation of the Sample

### 1 Desalting and N-Deglycosylation

In a first phase, the sample to be analyzed was salted out according to standard procedures so as to remove all the potentially present free reducing carbohydrates as well as the substances which may interfere during the subsequent steps (salts and excipients). After salting out, the sample was dried and then the glycans were released by enzymatic action of N-Glycanase under denaturation and reducing conditions, in order to maximize the yield of N-deglycosylation. For the N-deglycosylation of the Igs, the dry sample was taken up with 45 μL of the digestion solution PNGase F diluted to 1/5. 1.5 μL of a 10% (v/v) β-mercaptoethanol in ultra-pure water was added with stirring and incubation for 15 minutes at room temperature. Next, 1 μL of the PNGase F solution (2.5 mU/μL) was added before stirring and incubation in a water bath at 37° C. for 12 to 18 hours. Next the glycans were separated from the deglycosylated proteins by precipitation with cold EtOH.

The obtained glycan extract was then distributed into 4 fractions before being treated with exoglycosidases.

### 2 Quantification of the Fucosylation and Intercalating GlcNAc Levels, and of the Galactosylation Index of N-Glycans

Each dried alcoholic sub-fraction N, containing the equivalent of 100 μg of glycoprotein, were respectively digested (1) with α-sialidase, β-galactosidase and N-acetyl-β-hexosaminidase, in order to determine the fucosylation level; (2) with α-sialidase, β-galactosidase and α-fucosidase, for calculating the level of intercalating GlcNAc; and (3) with α-sialidase and α-fucosidase, for determining the galactosylation index.

These deglycosylations were carried out at 37° C. for 12 to 18 hours.

The isolation of the products of exoglycosidase degradations was achieved by cold alcohol extraction by adding 60 μL (3 volumes) of absolute ethanol equilibrated at −20° C., before stirring and then incubation at −20° C. for 15 minutes. Centrifugation at 10,000 rpm was achieved for 10 minutes at +4° C., and the supernatant was immediately transferred into a microtube of 0.5 mL before being dried in vacuo. The obtained oligosaccharides were then marked with a fluorochrome, APTS, and then separated and quantified in HPCE-LIF.

### 3 Utilization of the Results

The identification of the N-glycan peaks was achieved by means of a reference glycoprotein standard, the N-glycosylation of which is perfectly known, by comparison of the migration times of its N-glycans with those of the species observed on the electrophoretic profiles of the samples to be analyzed. Further, the migration times of the oligosaccharides standards are converted into glucose units (GUs) after analysis of a heterogeneous mixture of a glucose homopolymer (Glc ladder). These values of GUs will then be compared with those of a few standard oligosaccharides of known GUs, and will give the possibility of increasing the confidence level of the identifications.

### Results

A-Variants produced in YB2/0

The following polypeptides were analyzed:

| Name | Mutations |
|---|---|
| Anti-CD20 Del294 | Del294 |
| Anti-CD20-C6A_78-Del294 | T256N/A378V/S383N/N434Y/Del294 |
| Anti-CD20-C6A_74-Del294 | V259I/N315D/N434Y/Del294 |
| Anti-RhD | — |
| Anti-RhD Del294 | Del294 |
| Anti-RhD-C6A_78 | T256N/A378V/S383N/N434Y |
| Anti-RhD-C6A_78-Del294 | T256N/A378V/S383N/N434Y/Del294 |

### Anti-CD20 Del294:

The electropherograms obtained show biantenna glycan structures. These structures are in majority sialylated.

87.98% of the structures seem to be sialylated. The calculated fucosylation level is 48.24%.

| | |
|---|---|
| A2 | 11.9 |
| A2F | 19.8 |
| A1 | 5.5 |
| A1F | 1.6 |
| G0 | 0.84 |
| G0B | 0.96 |
| G1(1.6) + G0F | 0.53 |
| G1(1.3) + G0BF | 0.19 |
| G1(1.6)B | 2.44 |
| G1(1.6)F | 0.14 |
| G2 + G1(1.3)F | 2.09 |
| G2B | 0.51 |
| G2F | 0.16 |
| G2FB | 3.84 |
| Sialylated structures unidentified | 49.18 |
| % tage of sialylated structures: | 87.98 |
| Fucosylation % tage: | 48.24 |

### Anti-CD20-C6A 78-Del294:

The obtained electrophoregrams show biantenna glycan structures. These structures are in majority sialylated.

88.69% of the structures seem to be sialylated. The calculated fucosylation level is 51.87%

| | |
|---|---|
| A2 | 12.94 |
| A2F | 20.61 |
| A1 | 7.52 |
| A1F | 3.12 |
| G0 | 1.57 |
| G0B | 0.61 |
| G1(1.6) + G0F | 1.38 |
| G1(1.3) + G0BF | 0.58 |
| G1(1.6)B | 1.99 |
| G1(1.6)F | 0.51 |
| G2 + G1(1.3)F | 3.56 |
| G2B | 0.26 |
| G2F | 0.6 |
| G2FB | 0 |
| NI sialylated structures | 44.5 |
| % age of sialylated structures: | 88.69 |
| Fucoslylation % age: | 51.87 |

### Anti-CD20-C6A 74-Del294:

The obtained electrophoregrams show biantenna glycan structures. These structures are in majority sialylated.

93.48% of the structures seem to be sialylated. The calculated fucosylation level is 51.24%.

| | |
|---|---|
| A2 | 13.39 |
| A2F | 23.01 |
| A1 | 5.57 |
| A1F | 1.93 |
| G0 | 0.59 |
| G0B | 0.73 |
| G1(1.6) + G0F | 0.43 |
| G1(1.3) + G0BF | 0.18 |
| G1(1.6)B | 2 |
| G1(1.6)F | 0.11 |
| G2 + G1(1.3)F | 2.05 |
| G2B | 0.15 |
| G2F | 0.1 |
| G2FB | 0 |
| NI sialylated structures | 49.58 |
| % age of sialylated structures | 93.48 |
| Fucoslylation % age: | 51.24 |

### Anti-RhD:

The obtained electrophoregrams show biantenna glycan structures in majority consisting of short non-fucosylated agalactosylated short structures (G0: 52.06%). The fucosylated structures are a minority. A few structures having a GlcNac in a bissecting position (GOB, GOFB) are observed.

The predominant oligosaccharide structure is: G0 (52.06%). The calculated fucosylation level is 17.05%, the fucosylation level obtained with the run DSial+DGal+DhexNAc (*) is 13.07%. The level of forms having a bissecting GlcNac is 2.87%. The calculated galactosylation level is 40.5%.

| Structure (%) | HPCE-LIF |
|---|---|
| Sialylated | 0.00 |
| Mono-sialylated | 0.00 |
| Bi-sialvlated | 0.00 |
| Bissecting | 2.87 |
| Fucosylated* | 13.07 |
| Fucosylated | 17.05 |
| A2 | 0.00 |
| A2F | 0.00 |
| M3N2 | 0.00 |
| M3N2F | 0.00 |
| A1 | 0.00 |
| A1F | 0.00 |
| G2FB | 0.00 |
| G2F | 0.47 |
| G2B | 0.00 |
| G2 | 4.66 |
| G1FB | 0.00 |
| G1F | 5.65 |
| G1(1.3)FB | 0.00 |
| G1(1.6)FB | 0.00 |
| G1(1.3)F | 0.00 |
| G1(1.6)F | 5.65 |
| G1B | 0.00 |
| G1 | 24.59 |
| G1(1.3)B | 0.00 |
| G1(1.6)B | 0.00 |
| G1(1.3) | 4.00 |
| G1(1.6) | 20.59 |
| G0FB | 1.24 |
| G0F | 9.69 |
| G0B | 1.63 |
| G0 | 52.06 |
| MAN-5 | 0.00 |
| Identified (%) | 99.99 |

### Anti-RhD Del294:

The electrophoregrams obtained show biantenna glycan structures and a few triantenna structures. These structures are in majority sialylated.

92.25% of the structures seem to be sialylated. The calculated fucosylation level is 37.08%.

| | |
|---|---|
| A2 | 14.05 |
| A2F | 20.61 |
| A1 | 6.79 |
| A1F | 1.59 |
| G0 | 0.63 |
| G0B | 0.82 |
| G1(1.6) + G0F | 0 |
| G1(1.3) + G0BF | 0.72 |
| G1(1.6)B | 2.86 |
| G1(1.6)F | 0 |
| G2 + G1(1.3)F | 2.72 |
| G2B | 0 |
| G2F | 0 |
| G2FB | 0 |
| NI sialylated structures | 49.21 |
| % age of sialylated structures | 92 25 |
| Fucoslylation % age: | 37.08 |

### Anti-RhD -C6A 78:

The predominant oligosaccharide structure is: G0 (55.20%). The calculated fucosylation level is 12.37%, the fucosylation level obtained with the run DSial+DGal+DhexNAc (*) is 10.63%. The level of forms having a bissecting GlcNac is 2.27%. The calculated galactosylation level is 39.13%.

| Structure (%) | HPCE-LIF |
|---|---|
| Sialylated | 0.00 |
| Mono-sialvlated | 0.00 |
| Bi-sialylated | 0.00 |
| Bissecting | 2.27 |
| Fucosylated* | 10.63 |
| Fucosylated | 12.37 |
| A2 | 0.00 |
| A2F | 0.00 |
| M3N2 | 0.00 |
| M3N2F | 0.00 |
| A1 | 0.00 |
| A1F | 0.00 |
| G2FB | 0.00 |
| G2F | 0.00 |
| G2B | 0.00 |
| G2 | 4.00 |
| G1FB | 0.00 |
| G1F | 4.20 |
| G1(1.3)FB | 0.00 |
| G1(1.6)FB | 0.00 |
| G1(1.3)F | 0.00 |
| G1(1.6)F | 4.20 |
| G1B | 0.83 |
| G1 | 26.10 |
| G1(1.3)B | 0.00 |
| G1(1.6)B | 0.83 |
| G1(1.3) | 5.67 |
| G1(1.6) | 20.43 |
| G0FB | 0.66 |
| G0F | 7.51 |
| G0B | 1.50 |
| G0 | 55.20 |
| MAN-5 | 0.00 |
| Identified (%) | 100.00 |

### Anti-RhD -C6A 78-Del294:

The electrophoregrams obtained show biantenna glycan structures and a few triantenna structures. These structures are in majority sialylated.

91.83% of the structures seem to be sialylated. The calculated fucosylation level is 57.81%.

| | |
|---|---|
| A2 | 14.6 |
| A2F | 20.57 |
| A1 | 7.72 |
| A1F | 3.38 |
| G0 | 1.15 |
| G0B | 1.75 |
| G1(1.6) + G0F | 0 |
| G1(1.3) + G0BF | 0.62 |
| G1(1.6)B | 3.14 |
| G1(1.6)F | 0 |
| G2 + G1(1.3)F | 1.53 |
| G2B | 0 |
| G2F | 0 |
| G2FB | 0 |
| NI sialylated structures | 45.56 |
| % tage of sialylated structures: | 91.83 |
| Fucosylation % tage: | 57.81 |

### B-Variants Produced in the HEK Lines

The following polypeptides were analyzed (Anti-CD20 IgG variants):

| Name | Mutations |
|---|---|
| T5A-74 | N315D/A330V/N361D/A378V/N434Y |
| T5A-74H | V264E/N315D/A330V/N361D/A378V/N434Y |
| T5A-74Del294 | E294del/N315D/A330V/N361D/A378V/N434Y |
| WT | / |

The variant T5A-74H differs from the parent variant T5A-74 by the mutation V264E.

The mutant T5A-74De1294 differs from the parent variant T5A-74 by deletion of the amino acid in position 294.

The profile of glycosylation of these variants was subsequently analyzed. The results are summarized in the table hereafter (in percentages):

| | T5A-74 | T5A-74H | T5A-74Del294 | WT |
|---|---|---|---|---|
| A1 | 0 | 1.8 | 5.64 | 0 |
| A1F | 0 | 6.72 | 9.18 | 0 |
| Sialylated unidentified | 0 | 29.28 | 19.56 | 0 |
| G0 | 0 | 2.37 | 0 | 3.34 |
| G0B | 2.13 | 0.65 | 0 | 1.77 |
| G1(1.6) | 0 | 1 | 0 | 0 |
| G0F | 81.44 | 10.27 | 28.76 | 79.47 |
| G1(1.3) | 0 | 1.21 | 0 | 0 |
| G0FB | 1 | 0 | 4.92 | 0.88 |
| G1(1.6)B | 0.51 | 0 | 0 | 0.71 |
| G1(1.6)F | 10.08 | 12.15 | 6.64 | 9.23 |
| G2 | 0 | 1.85 | 0 | 0 |
| G1(1.3)F | 4 | 3.26 | 10.63 | 3.68 |
| G1(1.6)FB | 0 | 0 | 0 | 0 |
| G2B | 0 | 6.41 | 2 | 0 |
| G2F | 0.84 | 5.57 | 5.94 | 0.91 |
| G2FB | 0 | 1.65 | 1.57 | 0 |
| Galactosylation | 16.27 | >58.36 | >51.11 | 15.44 |
| Sialylation | 0 | >37.8 | >34.38 | 0 |
| Fucosylation | 97.36 | >40.88 | >67.64 | 94.17 |

### Example 3

### Analysis of the Half-Life of IgGs Deleted in Position 294

The persistence of immunoglobulins in the serum of transgenic mice for the human FcRn was evaluated. Two antigenic specificities were tested; the anti-CD20 IgGs and the anti-RhD IgGs deleted in position 294 were tested in comparison with the corresponding IgG WTs.

Pharmacokinetic experiments were thereby conducted in hFcRn mice which are homozygotes for an allele KO of the murine and heterozygote FcRn for a transgene of human FcRn (mFcRn^{-/-} hFcRnTg).

For these pharmacokinetic studies, each animal received a single intravenous injection of IgG at 5 mg/kg at the retro-orbital sinus, in a procedure similar to the one described earlier (Petkova SB, et al. Enhanced half-life of genetically engineered human IgG1 antibodies in a humanized FcRn mouse model: potential application in humorally mediated autoimmune disease. Int Immunol 2006). Blood samples were taken from the retro-orbital sinus at multiple points of time and the IgGs titrated with ELISA.

### Results:

In this test, both IgGs deleted in position 294 showed an increase in the half-life with a ratio (variant half-life/WT) of 1.7 (FIG. 2).

The analyzed parameters are grouped in the table below:

| | | | | removal | | |
|---|---|---|---|---|---|---|
| | C0 | AUC0-t | AUCinf | half-life | Vd | Cl |
| Molecules | (μg/mL) | (h · μg/mL) | (h · μg/mL) | (h) | (mL) | (mL/h) |
| Anti-CD20 WT | 121 | 1423 | 1425 | 29.2 | 4.07 | 0.097 |
| Anti-CD20 Del294 | 120 | 3482 | 3673 | 48.8 | 2.64 | 0.037 |
| Anti-RhDWT | 150 | 7491 | 8030 | 65.7 | 1.62 | 0.017 |
| Anti-RhD Del294 | 141 | 11142 | 13354 | 111 | 1.66 | 0.010 |

The analyzed parameters are defined below:
C0: Maximum concentration extrapolated to T0
AUCO−t: Area under the time/plasma concentration curve (from time T0 to the last time
t where the antibody is still quantifiable)
AUCinf: Area under the time/plasma concentration curve from T0 to infinity (=AUCO−t+extrapolation up to infinity)
T½: Half-life
Vd: Distribution volume
Cl: Clearance

### Example 4

### Production of Additional Fc Variants by Directed Mutagenesis and Binding Tests on the Fc Receptors:

### 1. Building Fc Variants:

Each mutation of interest in the fragment Fc was independently inserted into an expression vector containing the anti-CD20 heavy chain by overlap PCR by using two sets of primers adapted for integrating a deletion or a degenerate codon (NNN or NNK) in the targeted position (240 to 243, 258 to 267, 290 to 305). The fragments thereby obtained by PCR were associated and the resulting fragment was amplified by PCR by using standard procedures. The PCR product was purified on 1% (w/v) agarose gels, digested with suitable restriction enzymes and cloned in the eukaryotic expression vector pMGM05-CD20 (pCEP4 InvitroGen), which contains cloning sites for the Fc fragment (BamHl and Notl) and the variable chain VH of the anti-CD20 antibody. This construct causes mutation of two amino acids in the Fc (aa224 and 225, HT changed to GS) and the addition of the EFAAA sequence at the C-terminal of the Fc, but gives the possibility of very rapidly testing a very large number of clones. In a first phase, it was verified that these mutations do not modify the binding of IgG-WT to the different receptors. Subsequently, positive controls were cloned in this system in order to validate it:
IgG1-5239D, 1332E, from the anti-CD19 antibody XmAb5574 from Xencor (C1): positive control for CD16a;
IgG1-G236A, from Xencor (C4): positive control for CD32aH/R;
IgG1-K326W, E333S, from Abgenix/Genentech (C3): positive control for C1q; and
IgG1-5267E, L328F, from the anti-CD19 antibody XmAb5574 from Xencor (C5): positive control for CD32b.

The DNA of the isolated clones was sequenced after PCR on colonies. After bio-computer analyses, the clones including new mutations were frozen to −80° C. into a bacterium XL1-Blue and the sequences included in our database. Thus, 268 variants were built.

### 2. Production of IgG Variants in HEK293 Cells:

The light chain of the anti-CD20 was inserted into a pCEP4 vector identical with the vector used for the heavy chain, noted as pMGM01-CDC20 (pCEP4 InvitroGen). HEK293-F Freestyle™ (Invitrogen) cells, cultivated in 24-well plates, were co-transfected with the vectors pMGM01-CD20 and pMGM05-CD20 (Fc-WT and variants) in equimolar amounts (250 ng/ml) with a transfection reagent (1 μl/ml) by using standard procedures (Invitrogen). The cells were cultivated suspended in a medium without any serum for 7-9 days post-transfection and the supernatants (1 ml) containing IgGs were harvested after centrifugation of the cells at 100 G for 10 min. The IgGs secreted in the supernatants were quantified by using an ELISA test (FastELISA, R&D biotech).

### 3. Recombinant Fc Receptors Used:

CD16a is an activator receptor which has a polymorphism V/F in position 158, on the binding site to Fc. The affinity is better for CD16aV. CD16aV is commercially available (R&D system).

CD32a is an activator receptor which has a polymorphism H/R in position 131, on the binding site to Fc. The affinity is better for CD32aH. CD32aH was produced by PX′Therapeutics. CD32aR and CD32b are commercially available (R&D system).

### 4. ELISA Tests of IgG Variants Produced in the Supernatants of HEK293-F Cells:

The IgG variants were tested for their binding to several human FcRs and to the FcRn with ELISA. Maxisorp immunoplates were coated with 0.1 μg of CD32aH/well, or 0.2 μg CD16aV/well in PBS or 0.25 μg of FcRn in P6 (sodium phosphate 100 mM, sodium chloride 50 mM pH6.0). NiNTA plates (HisGrab Pierce) were coated with 0.05 μg of CD32aR/well, or 0.2 μg CD32b/well in PBS. After coating overnight at 4° C., the plates were washed twice with PBS (or P6)/0.05% Tween-20 and saturated with PBS/4% of BSA (or P6 4% in skimmed-milk) for 2 hours at 37° C. In parallel, the supernatants were diluted in PBS (or P6 for the test on FcRn) at a final concentration of 0.5 μg of IgG/ml and mixed with F(ab′)2 of anti-human goat HRP IgG at the same concentration for 2 hours at room temperature. The IgGs aggregated with the F(ab′)2 were then incubated with gentle stirring for 1 hour at 30° C. on saturated ELISA plates without any dilution for CD16aV, CD32aR and CD32b (i.e. IgG at 0.5 μg/ml), diluted in PBS at 0.25 pg/ml for CD32aH and diluted in P6 at 0.035 μg/ml for FcRn. The plates are then developed with TMB (Pierce) and the absorbence is read at 450 nm.

By means of this ELISA test, the built variants were tested in comparison with the wild type Fc (Fc-WT) and their variant/Fc-WT ratio was calculated, as indicated in tables 1 to 3.

1-18. (canceled)

19. A method for increasing the sialylation of an Fc fragment, comprising a mutation step of at least one amino acid selected from among amino acids in positions 240 to 243, 258 to 267 and 290 to 305 of said Fc fragment, the numbering being that of the EU index or equivalent in Kabat.

20. A method for producing a variant of a parent polypeptide comprising an Fc fragment, said variant having improved sialylation of said Fc fragment relatively to the sialylation of the Fc fragment of the parent polypeptide, which comprises a mutation step of at least one amino acid selected from among the amino acids in positions 240 to 243, 258 to 267 and 290 to 305 of said Fc fragment, the numbering being that of the EU index or equivalent in Kabat.

21. The method according to claim 19, wherein the sialylation of said Fc fragment is increased by at least 10% relatively to the sialylation of said Fc fragment before the mutation step or of said Fc fragment of the parent polypeptide.

22. A method for producing a variant of a parent polypeptide comprising an Fc fragment, said variant having at least one effector activity mediated by said Fc fragment reduced relatively to the effector activity of the parent polypeptide, further comprising a mutation step of at least one amino acid selected from among the amino acids in positions 240 to 243, 258 to 267 and 290 to 305 of said Fc fragment, the numbering being that of the EU index or equivalent in Kabat.

23. The method according to claim 22, wherein the effector activity mediated by the Fc fragment is selected from among cell cytotoxicity dependent on antibodies (ADCC), cytotoxicity depending on the complement (CDC) and cell phagocytosis dependent on the antibodies (ADCP).

24. The method according to claim 22, wherein the variant is without any effector activity mediated by the Fc fragment.

25. A method for producing a variant of a parent polypeptide comprising an Fc fragment, said variant having an affinity mediated by said Fc fragment reduced relatively to the affinity of the parent polypeptide, for at least one of the receptors of the Fc region (FcR), and the method comprising a mutation step of at least one amino acid selected from among the amino acids in positions 240 to 243, 258 to 267 and 290 to 305 of said Fc fragment, the numbering being that of the EU index or equivalent in Kabat.

26. The method according to claim 25, wherein the variant has an affinity mediated by said Fc fragment reduced relatively to the affinity of the parent polypeptide, for the receptor FcgRIIIa (CD16a) and the receptor FcgRIIa (CD32a).

27. The method according to claim 19, wherein the mutation is selected from among an insertion, a substitution, and a deletion.

28. The method according to claim 19, wherein the mutation is carried out on at least one amino acid located in position 240, 241, 242, 243, 258, 259, 260, 261, 262, 263, 264, 265, 266, 267, 290, 291, 292, 293, 294, 295, 296, 297, 298, 299, 300, 301, 302, 303, 304 or 305, the numbering being that of the EU index or equivalent in Kabat.

29. The method according to claim 19, wherein the mutation is carried out on at least one amino acid located in position 240, 241, 242, 243, 258, 259, 260, 261, 263, 265, 266, 267, 290, 291, 292, 293, 294, 295, 296, 298, 299, 300, 301, 302, 303, 304 or 305.

30. The method according to claim 19, wherein the mutation is carried out on the amino acid located in position 293 or 294, the numbering being that of the EU index or equivalent in Kabat.

31. The method according to claim 19, wherein the Fc fragment of the parent polypeptide already comprises at least one additional mutation, selected from among a combination of additional mutations selected from among P230S/N315D/M428L/N434Y, T256N/A378V/S383N/N434Y, V2591/N315D/N434Y and N315D/A330V/N361D/A378V/N434Y.

32. The method according to claim 20, wherein the parent polypeptide consists in an Fc fragment.

33. The method according to claim 20, wherein the parent polypeptide consists in a sequence of amino acids fused in the N- or C-terminal to an Fc fragment.

34. The method according to claim 20, wherein said parent polypeptide is an immunoglobulin or an antibody.

35. The method according to claim 19, wherein the Fc fragment of the parent polypeptide is an Fc fragment of an IgG1, corresponding to the sequence SEQ ID NO: 1.

36. The method according to claim 20, wherein the mutation step is obtained as follows:
i) a nucleic acid sequence coding for the parent polypeptide comprising the Fc fragment is provided;
ii) the nucleic sequence provided in i) is modified in order to obtain a nucleic sequence coding for the variant; and
iii) the nucleic sequence obtained in ii) is expressed in a host cell, and the variant is recovered.

37. The method of claim 25, wherein the at least one of the receptors is selected from among the complement C1q and the receptors FcgRIIIa (CD16a), FcgRIIa (CD32a), and FcgRIIb (CD32b).

38. The method of claim 27, wherein the substitution is point like.


SEQUENCE LISTING (10 sequences)

SEQ ID NO: 1 - protein, 222 aa, Artificial Sequence
  Fc de l′IgG1 G1m1,17
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 221 aa, Artificial Sequence
  Fc d′ IgG2
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 222 aa, Artificial Sequence
  Fc d′ IgG3
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 222 aa, Artificial Sequence
  Fc d′ IgG4
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 222 aa, Artificial Sequence
  Fc de l′IgG1 G1m3
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Fc de l′IgG1 G1m1,17
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Artificial Sequence
  Fc d′ IgG2
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Artificial Sequence
  Fc d′ IgG3
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Artificial Sequence
  Fc d′ IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 232 aa, Artificial Sequence
  Fc de l′IgG1 G1m3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK